(19) United States
(12) Patent Application Publication
(10) Pub. No.: US 2026/0115146 A1
(43) Pub. Date: Apr. 30, 2026

(54) WATER-SOLUBLE NANO-COMPLEX WITH PHYTOCHEMICAL

(71) Applicant: Nano and Advanced Materials Institute Limited, Hong Kong (HK)

(72) Inventors: Chun Hay KO, Hong Kong (HK); Chun Fai NG, Hong Kong (HK); Ying Ying NG, Hong Kong (HK); Weiyi SHE, Hong Kong (HK)

(21) Appl. No.: 19/243,010

(22) Filed: Jun. 19, 2025

Related U.S. Application Data
(60) Provisional application No. 63/713,569, filed on Oct. 29, 2024.

(51) Int. Cl. A61K 9/51; A61K 31/12; A61K 31/352; A61K 31/353; A61K 31/704; A61K 36/82

(52) U.S. Cl. CPC A61K 9/5192; A61K 9/5115; A61K 9/5123; A61K 9/5169; A61K 31/12; A61K 31/352; A61K 31/353; A61K 31/704; A61K 36/82

(57) ABSTRACT

A method for encapsulating a low-solubility phytochemical into a water-soluble nano-complex is presented. The method involves creating a mixture of one or more proteins and a solvent, followed by adjusting the pH to an alkaline condition using an alkaline compound. Zein protein and a low-solubility phytochemical are sequentially added, with the proteins unfolding under alkaline conditions to capture the phytochemical through hydrophobic interactions. The pH is then adjusted to a neutral range using an acidic compound, refolding the zein protein and forming a nano-complex. The solution is dried to produce a dry powder, where the resulting nano-complex has a particle size of less than 1 μm, with the proteins encapsulating the phytochemical.

### CROSS-REFERENCE TO RELATED APPLICATIONS

The present application claims priority from U.S. Provisional Utility Patent application No. 63/713,569 filed Oct. 29, 2024; the disclosure of which is incorporated herein by reference in its entirety.

### FIELD OF THE INVENTION

The present invention generally relates to pharmaceutical sciences and biotechnology field. More specifically the present invention relates to methods for encapsulating a low-solubility phytochemical to form a water-soluble nano-complex.

### BACKGROUND OF THE INVENTION

In the increasingly health-conscious world, consumers are gravitating toward natural and safe alternatives to synthetic drugs. Active pharmaceutical ingredients (APIs) derived from plants, known as phytochemicals, have played a significant role in traditional medicine for centuries and continue to be a vital component of modern health practices. These plant-derived compounds offer a range of health benefits, including antioxidant, anti-inflammatory, antimicrobial, and even potential anti-cancer properties. Due to their natural origin and multiple biological effects, phytochemicals have become a cornerstone of nutraceuticals and functional foods, catering to the growing demand for health-conscious and preventive healthcare solutions.

However, despite their therapeutic potential, the clinical and commercial application of phytochemicals remains limited due to several significant challenges, particularly their low bioavailability. Many of these beneficial compounds are hydrophobic, which leads to poor water solubility and difficulties in absorption when ingested. In addition, rapid metabolism and elimination from the body further reduce their efficacy. The large particle size of hydrophobic molecules exacerbates these challenges, hindering the efficient delivery of these compounds to the target tissues, thus limiting their therapeutic potential.

Extensive research has been conducted to enhance the bioavailability of phytochemicals by creating bioavailable complexes, such as encapsulation in nanoparticles, liposomes, and other drug delivery systems. While these innovations hold promise, the commercialization of such systems as health supplements is still fraught with difficulties. A major obstacle is the use of toxic organic solvents and synthetic surfactants in the production process. These chemicals, while effective in solubilizing and stabilizing hydrophobic compounds, raise significant safety and regulatory concerns, particularly when intended for human consumption. Additionally, many of these phytochemical complexes encounter challenges during manufacturing, such as the inability to re-disperse effectively in water after the drying process, which is essential for achieving consistent bioavailability in oral dosage forms.

As a result, the development of a new technology that addresses these challenges is critical to unlocking the full potential of plant-based APIs. By creating a system that enhances the bioavailability of phytochemicals, eliminates the need for toxic solvents and surfactants, and ensures stability and re-dispersibility in water, the commercialization of these valuable health-promoting compounds could be significantly advanced. This would pave the way for more effective, natural, and safe alternatives to conventional pharmaceuticals, meeting the demands of an increasingly health-conscious population. Therefore, the present invention addresses this need.

### SUMMARY OF THE INVENTION

It is an objective of the present invention to provide the compound, material, and method to solve the aforementioned technical problems.

In accordance with a first aspect of the present invention, a method for encapsulating a low-solubility phytochemical to form a water-soluble nano-complex is provided. The method includes the following steps: creating a mixture by combining one or more proteins and a solvent; adjusting the pH of the mixture to an alkaline condition by adding an alkaline compound; sequentially adding zein protein and a low-solubility phytochemical into the mixture with mixing, wherein the zein protein unfolds under the alkaline condition, allowing the zein protein to capture the low-solubility phytochemical through hydrophobic interactions; adjusting the pH of the mixture to an approximately neutral pH value by adding an acidic compound, thereby refolding the proteins to fix the low-solubility phytochemical and forming a nano-complex solution; and drying the nano-complex solution to produce a dry powder form of the nano-complex.

In accordance with one embodiment of the present invention, the nano-complex includes the one or more proteins encapsulating the low-solubility phytochemical, and the nano-complex has a particle size less than 1 μm.

In accordance with another embodiment of the present invention, the method further includes a step of heating the mixture to a temperature of 80-99° C. before the addition of the low-solubility phytochemical and zein protein.

In accordance with yet another embodiment of the present invention, the heating is applied for a duration of 5 to 30 minutes.

In accordance with yet another embodiment of the present invention, the low-solubility phytochemical includes one or more of curcumin, quercetin, ginsenosides, tea polyphenols, and tetrahydrocurcumin.

In accordance with yet another embodiment of the present invention, the one or more proteins are water-soluble and selected from animal proteins, plant-based proteins or a combination thereof.

In accordance with yet another embodiment of the present invention, the alkaline compound is sodium hydroxide, potassium hydroxide or a combination thereof.

In accordance with yet another embodiment of the present invention, the alkaline condition is a condition with a pH value greater than 11.

In accordance with yet another embodiment of the present invention, the approximately neutral pH value is a pH value ranges from 5 to 8.

In accordance with yet another embodiment of the present invention, the acidic compound is hydrochloric acid, citric acid, ascorbic acid, lactic acid, malic acid, acetic acid or a combination thereof.

In accordance with yet another embodiment of the present invention, the drying of the nano-complex solution to obtain the dry powder is performed using freeze drying, convection drying, or spray drying.

In accordance with yet another embodiment of the present invention, the protein encapsulation increases the solubility of the low-solubility phytochemical by at least 150%.

In accordance with yet another embodiment of the present invention, the nano-complex has a particle size ranging from 40 to 200 nanometers.

In accordance with yet another embodiment of the present invention, the one or more proteins are selected from the group consisting of casein, whey protein, soy protein, and pea protein.

In accordance with a second aspect of the present invention, a water-soluble encapsulated phytochemical complex powder fabricated by the aforementioned method is introduced. The powder includes the water-soluble nano-complex. Particularly, the water-soluble nano-complex has an encapsulation layer, including the one or more proteins; and a core, including the zein protein and the low-solubility phytochemical. It is worth noting that the zein protein and the low-solubility phytochemical are bound together through hydrophobic interactions and the water-soluble nano-complex has a particle size less than 1 μm.

In accordance with one embodiment of the present invention, the particle size of the water-soluble nano-complex ranges from 40 to 200 nanometers.

In accordance with another embodiment of the present invention, the encapsulated phytochemical exhibits increased water solubility and bioavailability compared to its unencapsulated form.

In accordance with yet another embodiment of the present invention, the low-solubility phytochemical includes one or more of curcumin, quercetin, ginsenosides, tea polyphenols, and tetrahydrocurcumin.

In accordance with yet another embodiment of the present invention, the encapsulation layer is formed from a combination of plant-based proteins and animal-based proteins.

In accordance with yet another embodiment of the present invention, the encapsulated phytochemical is used in pharmaceutical, nutraceutical, cosmetic or food supplement applications.

In accordance with yet another embodiment of the present invention, the encapsulation layer provides physical protection and oxidative stability to the low-solubility phytochemical.

### BRIEF DESCRIPTION OF THE DRAWINGS

Embodiments of the invention are described in more details hereinafter with reference to the drawings, in which:

FIG. 1 depicts a water-soluble nano-complex including protein encapsulated phytochemicals in according to one embodiment of the present invention;

FIGS. 2A-2D depict the cytotoxicity of F1 (FIG. 2A), F2 (FIG. 2B); F3 (FIG. 2C) and F4 (FIG. 2D) on Caco-2 cells. Cell viability is determined by MTT assay;

FIGS. 3A-3D depict the anti-inflammatory effect of sample F1 on RAW 264.7 cells, in which FIG. 3A shows the cell viability of different concentrations of F1 by MTT assay; FIG. 3B displays that NO production upon F1 and unformulated control treatment determined by Griess assay; FIG. 3C shows the ELISA results of interleukin 6 expression upon F1 and unformulated control treatment; and FIG. 3D depicts the ELISA assay of prostaglandin E₂ (PGE₂) expression upon F1 and unformulated control treatment;

FIGS. 4A-4D depict the anti-inflammatory effect of sample F3 on RAW 264.7 cells, in which FIG. 4A shows the cell viability of different concentrations of F3 by MTT assay; FIG. 4B displays that NO production upon F3 and unformulated control treatment determined by Griess assay; FIG. 4C shows the ELISA results of interleukin 6 expression upon F3 and unformulated control treatment; and FIG. 4D depicts the ELISA assay for PGE₂ expression upon F3 and unformulated control treatment;

FIGS. 5A-5C depict the anti-adipogenic effect of sample F2 on 3T3-L1 cells, in which FIG. 5A shows the effects of F2 and unformulated control on the differentiation of 3T3-L1 cells by Oil Red O staining (Magnification: 200×); FIG. 5B depict the cell viability of different concentrations of F2 on 3T3 cells; and FIG. 5C depicts the relative lipid contents upon F2 and unformulated control treatment; and

FIGS. 6A-6C depict the anti-adipogenic effect of sample F4 on 3T3-L1 cells; FIG. 6A shows the effects of F4 and unformulated control on the differentiation of 3T3-L1 cells by Oil Red O staining (Magnification: 200×); FIG. 6B shows the cell viability of different concentrations of F4 on 3T3 cells; and FIG. 6C displays the relative lipid contents upon F4 and unformulated control treatment.

### DETAILED DESCRIPTION

In the following description, methods of encapsulating low-solubility phytochemicals to create a water-soluble nano-complex and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The term “phytochemical” refers to naturally occurring compounds found in plants that contribute to their color, flavor, and defense mechanisms. These compounds, while not essential nutrients like vitamins or minerals, have been shown to offer health benefits. Phytochemicals include a wide variety of substances such as flavonoids, carotenoids, polyphenols, and alkaloids, many of which possess antioxidant, anti-inflammatory, and anti-cancer properties. They are commonly found in fruits, vegetables, grains, and other plant-based foods, and are believed to play a role in reducing the risk of chronic diseases like heart disease, diabetes, and certain cancers.

The term “low-solubility phytochemicals” used herein refers to plant-derived compounds that exhibit poor solubility in water or biological fluids, making them challenging to absorb in the body when consumed. These compounds often possess hydrophobic properties, meaning they do not readily dissolve in aqueous environments, limiting their bioavailability and therapeutic efficacy. Examples of difficult-to-dissolve phytochemicals include curcumin, resveratrol, quercetin, and certain flavonoids. To enhance their bioavailability, various formulation strategies, such as nano-encapsulation, emulsification, or the use of solubilizing agents, are often employed.

In accordance with a first aspect, the present invention provides a method for encapsulating low-solubility phytochemicals to create a water-soluble nano-complex, enhancing the bioavailability of these compounds. The process begins by forming a mixture, achieved by combining one or more proteins with a solvent. To optimize the environment for encapsulation, the pH of the mixture is adjusted to an alkaline condition through the addition of an alkaline compound, such as sodium hydroxide, potassium hydroxide or other strong bases. This results in a pH level above 11, which is critical for the subsequent steps of the process.

The alkaline treatment in the encapsulation process is crucial as it can significantly improve the solubility of plant-based proteins and unfold protein's tertiary structures for phytochemicals encapsulation. Apart from these, this treatment makes the difficult-to-dissolve phytochemicals dissolve in aqueous alkaline solution, which is a very important step as the phytochemicals should be fully dissolved in solution for the encapsulation process. Then, the hydrophobic phytochemicals can be attracted by the hydrophobic amino acids of the unfolded proteins, assisting the encapsulation process. Given the significance of alkaline treatment, it is essential to optimize the treatment duration. Various proteins or phytochemicals require distinct treatment times to achieve their optimal encapsulation performance, and normally the treatment duration is around 5 to 30 mins.

Once the alkaline condition is established, the method proceeds by sequentially adding zein protein into the mixture. Following this, a low-solubility phytochemical, such as curcumin, quercetin, ginsenosides, tea polyphenols, or tetrahydrocurcumin, is also introduced. Under the alkaline conditions, the zein protein unfolds, creating a hydrophobic environment that allows it to interact with and capture the hydrophobic low-solubility phytochemical. This hydrophobic interaction is key to the successful encapsulation of the phytochemical.

After the zein protein has captured the phytochemical, the pH of the mixture is adjusted back to an approximately neutral range, typically between pH 5 and 8, by adding an acidic compound to form edible salts. Suitable acids for this step include hydrochloric acid, citric acid, ascorbic acid, lactic acid, malic acid, or acetic acid. This pH adjustment causes the proteins to refold, effectively securing the phytochemical within its structure and forming a stable nano-complex solution. In this refolded state, the hydrophobic amino acids collapse inward, shielding themselves from the aqueous environment. As a result, the hydrophobic phytochemicals are encapsulated inside the refolded protein due to the attraction between the hydrophobic amino acids, thereby creating a stable nano-complex solution.

The nano-complex solution is then subjected to a drying process to convert it into a dry powder form. The drying can be performed using various techniques, including freeze drying, convection drying, or spray drying, depending on the desired characteristics of the final product. The resulting nano-complex includes the one or more proteins encapsulating the phytochemical, thereby creating a stable, water-soluble powder that can significantly increase the solubility of the phytochemical—by at least 150% compared to its unencapsulated form. Particularly, the particle size of the nano-complex is less than 1 μm.

In some embodiments of the method, the mixture is heated to a temperature of 80-99° C., which above the protein denaturation temperatures, before the addition of the phytochemical and zein protein. This heating step, which lasts for 5 to 30 minutes, can improve the interaction between the components and enhance the overall encapsulation efficiency.

In some embodiments, the mixing method includes mechanical stirring, ultrasound treatment, and homogenization.

The nano-complex formed by this method has a particle size preferably less than 1 μm, more preferably, less than 500 nm, and even more preferably 40-200 nm, providing optimal solubility and stability. The use of water-soluble proteins, which can be selected from either animal-based or plant-based sources, further enhances the versatility and potential applications of this encapsulation system. In some embodiments, the proteins may be some animal proteins, such as whey protein and fish protein, or plant-based proteins such as soy protein, pea protein and rice protein, or a mixture of the animal and plant-based proteins.

It is important to note that many plant-based proteins exhibit poor water solubility, which significantly impacts both the solubility and particle size of the encapsulated phytochemicals. Thermal treatment and solvent-based methods used for protein extraction can alter protein solubility by denaturing the proteins and exposing hydrophilic amino acid residues. To address this, solubility can be improved by applying heat treatment to protein dispersions above their denaturation temperatures under alkaline conditions. This is followed by neutralization, resulting in a stable dispersion at neutral or near-neutral pH levels, which enhances the encapsulation process.

In contrast, animal proteins, such as whey protein, generally have better water solubility than plant-based proteins. However, as macromolecules, the functional properties of proteins are determined by the specific composition, sequence, and length of their amino acid residues. Consequently, the compatibility between a phytochemical and a native protein may not be sufficient to achieve the desired performance. To optimize protein complexation with specific phytochemicals, structural modifications are performed on selected proteins using alkaline treatment. This treatment unfolds the protein's tertiary structure, facilitating the encapsulation process by improving the interaction between the protein and the phytochemical.

In some embodiments, the one or more proteins may include casein, whey protein, soy protein, pea protein or a combination thereof.

Overall, this method offers a sustainable, effective solution for increasing the bioavailability of low-solubility phytochemicals, allowing them to be used more effectively in pharmaceutical, nutraceutical, cosmetic and food supplement products.

In accordance with a second aspect, the present invention further introduces a water-soluble encapsulated phytochemical complex powder, which is fabricated using the method previously mentioned. The water-soluble encapsulated phytochemical complex powder includes a water-soluble nano-complex having two distinct components: an encapsulation layer and a core. The encapsulation layer is formed from one or more proteins, providing structural integrity and stability to the complex. Within the core, the low-solubility phytochemical is bound to zein protein, a corn-derived protein known for its hydrophobic properties. This binding is facilitated through hydrophobic interactions, ensuring the stable encapsulation of the low-solubility phytochemical.

The particle size of the nano-complex preferably less than 1 μm, more preferably, less than 500 nm, and even more preferably 40-200 nm. This nano-scale size enhances the solubility and bioavailability of the encapsulated phytochemical, significantly improving its efficacy compared to its unencapsulated form. By reducing the particle size to the nanometer range, the complex becomes more easily dispersed in aqueous solutions, allowing for more efficient delivery and absorption when consumed.

The phytochemicals encapsulated in this complex may include, but are not limited to, curcumin, quercetin, ginsenosides, tea polyphenols, tetrahydrocurcumin or combinations thereof. These phytochemicals, known for their health-promoting properties, typically face limitations due to their low water solubility. The encapsulation system described here addresses this challenge, enhancing their solubility and allowing them to be used effectively in various applications.

The encapsulation layer, which surrounds the phytochemical core, can be made from a combination of plant-based and animal-based proteins. This dual-protein approach not only improves the structural properties of the encapsulation but also increases the versatility of the complex, making it suitable for a broader range of formulations. The selection of proteins can be tailored based on the desired characteristics of the final product, whether for enhanced stability, biocompatibility, or solubility. It is worth noting that the encapsulation layer also provides physical protection and oxidative stability to the low-solubility phytochemical.

The water-soluble encapsulated phytochemical complex powder is highly versatile and can be used in various applications, particularly in the pharmaceutical, nutraceutical, cosmetic and food supplement industries. In these fields, the enhanced solubility and bioavailability of the encapsulated phytochemicals make the complex an ideal ingredient for developing products aimed at promoting health and wellness. By improving the delivery and efficacy of low-solubility phytochemicals, this innovative complex opens new possibilities for their practical application in consumer products.

In summary, the invention provides a new perspective on transforming insoluble phytochemicals into water-soluble, nano-sized complexes using a sustainable and environmentally friendly process. By encapsulating these compounds within protein-based nanoparticles, their solubility and bioavailability are significantly enhanced. This breakthrough technology offers a promising solution to bridging the gap between the health-promoting potential of natural compounds and their practical application in healthcare.

The protein nano-complexation concept has shown an excellent safety profile. In vitro studies indicate no cytotoxicity, while in vivo experiments confirm the absence of toxicity within reasonable dosages. Furthermore, this novel formulation enhances intestinal absorption and delivers significant biological effects, demonstrating its potential as a safe and effective delivery system.

In addition to addressing the challenges associated with phytochemical delivery, this protein nano-complexation strategy aligns with sustainable practices. By avoiding the use of toxic solvents and synthetic additives, the strategy prioritizes both human health and environmental protection. These protein-based complexes serve as safe carriers, helping valuable phytochemicals reach their intended targets in the body.

### EXAMPLES

### Example 1. Structure of the Encapsulated Phytochemicals

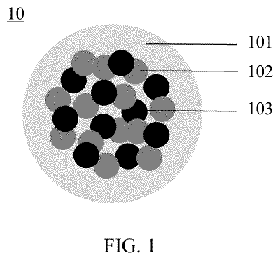

As shown in FIG. 1, a water-soluble nano-complex 10 containing phytochemicals, according to one embodiment of the present invention, is depicted. Zein protein 102 contains numerous hydrophobic amino acids, which facilitate strong hydrophobic interactions between the zein protein 102 and the phytochemicals 103. Under alkaline conditions, the structure of the zein protein 102 unfolds, exposing its hydrophobic amino acids. These exposed hydrophobic regions interact with the phytochemicals 103, leading to their encapsulation. Once the pH is neutralized, the zein protein 102 refolds, effectively encapsulating the phytochemicals 103 within its structure.

The encapsulating proteins 101, which form the outer layer of the nano-complex, contain a higher proportion of hydrophilic amino acids with fewer hydrophobic residues, making them soluble in water. In the case of modified plant-based proteins, the hydrolyzed peptide chains are shorter compared to their unmodified counterparts, enhancing their water solubility. Whether using animal proteins or modified plant-based proteins, their amphiphilic structures play an important role in the encapsulation process. The hydrophilic groups face outward, dissolving in water, while the hydrophobic groups are hidden inside, interacting with either the zein protein or the encapsulated phytochemicals. These amphiphilic and water-soluble proteins form the outer protective layer of the encapsulated product.

### Example 2. Fabrication of the Water-Soluble Nano-Complex Containing Encapsulated Phytochemicals

### Encapsulation of Curcumin Using Hydrolyzed Soy Protein and Zein Protein

To prepare the nano-complex, 24 grams of hydrolyzed soy protein are dissolved in 861.6 mL of deionized water under continuous stirring with an electric stirrer for 5 minutes. Subsequently, 60 mL of 1 M sodium hydroxide solution is added to the mixture to achieve an alkaline condition, and stirring is continued for an additional 5 minutes. The resulting solution is then subjected to heat treatment by placing the container in a 90° C. water bath for 5 minutes. After heating, the mixture is rapidly cooled by immersing the container in a room-temperature or ice water bath until the temperature drops below 35° C. Once cooled, 2.4 grams of zein protein are added, and the solution is stirred for 10 minutes to promote protein unfolding and dispersion. This is followed by the addition of 3.6 grams of curcumin, which is mixed under dark conditions for another 10 minutes to protect the compound from photodegradation. Finally, the pH of the mixture is adjusted to 6.5 by the gradual addition of 0.5 M citric acid solution, enabling zein refolding and nano-complex formation. The resulting suspension is then freeze-dried under dark conditions to obtain a dry powder product, designated as sample F1.

### Encapsulation of Quercetin and Ginsenoside Rg1 Using Hydrolyzed Soy Protein and Zein Protein

To prepare the nano-complex formulation, 36 grams of hydrolyzed soy protein are first dissolved in 861.6 mL of deionized water in a container and stirred using an electric stirrer for 5 minutes to ensure complete dispersion. Following this, 60 mL of 1 M sodium hydroxide solution is added to alkalize the mixture, which is then stirred for an additional 5 minutes. The solution is subsequently heated by placing the container in a 90° C. water bath for 10 minutes to facilitate protein unfolding. After heating, the mixture is promptly cooled by immersing the container in a room-temperature or ice water bath until the temperature falls below 35° C. Once cooled, 3.6 grams of zein protein are introduced and stirred for 10 minutes to allow proper dispersion and interaction under alkaline conditions. This is followed by the sequential addition of 2.1 grams of Trolox, stirred for 2 minutes, and the simultaneous addition of 2.1 grams of ginsenoside and 2.1 grams of quercetin, with the mixture stirred in the dark for 10 minutes to prevent light-induced degradation of the phytochemicals. The pH of the solution is then adjusted to 7.0 by the gradual addition of 0.5 M citric acid solution, enabling the refolding of zein and encapsulation of the bioactive compounds. The resulting nano-complex solution is freeze-dried in the dark, and the final dry powder is collected and designated as sample F2.

### Encapsulation of Curcumin Using Whey Protein Isolate and Zein Protein

In another formulation procedure, 32.4 grams of whey protein isolate are dissolved in 861.6 mL of deionized water and stirred using an electric stirrer for 30 minutes to ensure complete dissolution. Subsequently, 60 mL of 1 M sodium hydroxide solution is added to the mixture, which is then stirred for an additional 5 minutes to achieve alkaline conditions. Next, 3.6 grams of zein protein are incorporated and stirred for 10 minutes, followed by the addition of 3.6 grams of curcumin. The mixture is stirred in the dark for 30 minutes to prevent degradation of curcumin. The pH of the solution is then adjusted to 6.5 by the gradual addition of 0.5 M citric acid solution. The final mixture is freeze-dried in the dark, and the resulting dry powder is collected and designated as sample F3.

### Encapsulation of Quercetin Using Whey Protein Isolate and Zein Protein

To prepare a quercetin nano-complex, 32.4 grams of whey protein isolate are dissolved in 861.6 mL of deionized water and stirred for 30 minutes using an electric stirrer. Then, 60 mL of 1 M sodium hydroxide solution is added and stirred for an additional 5 minutes. Following alkalization, 3.6 grams of zein protein are added and stirred for 10 minutes. Next, 1.08 grams of quercetin are introduced, and the mixture is stirred in the dark for 5 minutes. The pH is adjusted to 7.0 using 0.5 M citric acid solution added dropwise. The mixture is freeze-dried in the dark, and the resulting dry powder is collected as sample F4.

### Encapsulation of Ginsenoside Rg1 Using Whey Protein Isolate and Zein Protein

In another formulation, 32.4 grams of whey protein isolate are dissolved in 861.6 mL of deionized water and stirred with an electric stirrer for 30 minutes. Then, 60 mL of 1 M sodium hydroxide solution is added and stirred for an additional 5 minutes. Afterward, 3.6 grams of zein protein are added to the alkaline solution and stirred for 10 minutes, followed by the addition of 9.0 grams of ginsenoside Rg1. The mixture is stirred in the dark for 5 minutes. The pH is then adjusted to 7.0 by gradually adding 0.5 M citric acid solution. The final solution is freeze-dried in the dark, and the resulting powder is collected as sample F5.

### Encapsulation of Tea Polyphenol Using Whey Protein Isolate and Zein Protein

To encapsulate tea polyphenols, 32.4 grams of whey protein isolate are dissolved in 861.6 mL of deionized water and stirred for 30 minutes with an electric stirrer. Next, 60 mL of 1 M sodium hydroxide solution is added and stirred for 5 minutes. Then, 3.6 grams of zein protein are incorporated and stirred for 10 minutes, followed by the addition of 0.2 grams of tea polyphenol. The mixture is stirred in the dark for 5 minutes. The pH is adjusted to 7.0 using 0.5 M citric acid solution. The final mixture is freeze-dried in the dark, and the resulting dry powder is collected and labeled as sample F6.

### Encapsulation of Epigallocatechin Gallate (EGCG) Using Whey Protein Isolate and Zein Protein

In a similar preparation, 32.4 grams of whey protein isolate are dissolved in 861.6 mL of deionized water and stirred for 30 minutes using an electric stirrer. Then, 60 mL of 1 M sodium hydroxide solution is added and stirred for 5 minutes. After this step, 3.6 grams of zein protein are added and stirred for 10 minutes, followed by the addition of 0.2 grams of epigallocatechin gallate (EGCG), with continued stirring in the dark for 5 minutes. The pH of the solution is adjusted to 7.0 by slowly adding 0.5 M citric acid solution. The mixture is then freeze-dried in the dark, and the resulting dry powder is collected as sample F7.

### Encapsulation of Tetrahydrocurcumin Using Whey Protein Isolate and Zein Protein

In another procedure, 32.4 g of whey protein isolate is dissolved in 861.6 mL of deionized water and stirred for 30 minutes using an electric stirrer. Afterward, 60 mL of 1M sodium hydroxide solution is added and stirred for 5 minutes. Then, 3.6 g of zein protein is incorporated into the solution and stirred for 10 minutes, followed by the addition of 1.8 g of tetrahydrocurcumin, which is stirred in the dark for 30 minutes. Finally, 0.5M citric acid solution is added gradually to adjust the pH to 6.5. The mixture is freeze-dried in the dark and collected as a dry powder (sample F8).

### Particle Size and Encapsulation Efficiency Measurements

Respectively dissolving 0.01 g of samples F1-F8 in 3 mL of water and mixed by vortex mixer. The solution is subjected to analysis by Dynamic Light Scattering (DLS), so as to measure the particle size. The mean particle size and PDI for each formulation is analyzed by 3 times (n=3). The results are shown in table 1.

Additionally, the samples are extracted with 70% ethanol and ultrasound for 20 min. The sample solution is then analyzed by high-performance liquid chromatography (HPLC) to determine the concentrations of phytochemicals in samples.

The encapsulation efficiency is calculated as follow:

$$Encapsulation efficiency = \frac{Found concentration of chemical marker in prototype}{Theorectical concentration of chemical marker in prototype} \times 100 \%$$

The loading of phytochemicals is calculated as follow:

$$Loading of phytochemical = \frac{Found amount of chemical marker in prototype}{Sample weight of prototype} \times 100 \%$$

The mean encapsulation efficiency and the mean loading of phytochemicals in each formulation are analyzed by 3 times (n=3). The results are shown in table 1.

TABLE 1
The particle size, encapsulation efficiency and
loading of phytochemicals in each formulation

| | Particle | | | |
|---|---|---|---|---|
| | size analysis | | Encapsulation | Loading of |
| Formu- | Particle | | efficiency | phytochemicals |
| lation | size (nm) | PDI | (%) | (%) |
| F1 | 192.0 | 0.47 | Curcumin: 80.78 | Curcumin: 6.96 |
| F2 | 94.9 | 0.17 | Quercetin: 78.86 | Quercetin: 3.21 |
| | | | Ginsenoside | Ginsenoside |
| | | | Rg1: 90.64 | Rg1: 2.31 |
| F3 | 146.4 | 0.35 | Curcumin: 90.48 | Curcumin: 5.22 |
| F4 | 74.5 | 0.45 | Quercetin: 89.25 | Quercetin: 1.97 |
| F5 | 89.3 | 0.37 | Ginsenoside | Ginsenoside |
| | | | Rg1: 88.95 | Rg1: 9.49 |
| F6 | 49.3 | 0.38 | EGCG: 94.96 | EGCG: 4.54 |
| F7 | 50.62 | 0.40 | EGCG: 85.23 | EGCG: 10.34 |
| F8 | 82.74 | 0.35 | Tetrahydro- | Tetrahydro- |
| | | | curcumin: 83.51 | curcumin: 3.12 |

### Comparative Example 1: Preparation of Unformulated Curcumin in Aqueous Solution

This comparative example serves as the unencapsulated control for Sample F1. Specifically, 9.04 mg of raw curcumin (with a purity of 77%) is added to 20 mL of Dulbecco's Modified Eagle Medium (DMVEM). Despite extensive mixing by both magnetic stirring and vortex agitation, the curcumin remains visibly undissolved in the medium, indicating poor aqueous solubility. The resulting suspension is diluted 40-fold prior to its use in subsequent in vitro anti-inflammatory assays.

Similarly, for the unformulated control corresponding to Sample F3, 7.14 mg of raw curcumin (77% purity) is added to 20 mL of DMEM. Despite identical attempts to facilitate solubilization via stirring and vortexing, the curcumin remains largely insoluble. The undissolved mixture is also diluted 40-fold for subsequent in vitro evaluation.

### Comparative Example 2: Preparation of Unformulated Quercetin and Ginsenoside Rg1 in Aqueous Solution

As the control for Sample F2, a mixture of 7.24 mg of quercetin (88.67% purity) and 8.33 mg of ginsenoside Rg1 (55.46% purity) is added to 10 mL of DMEM. Despite agitation through stirring and vortexing, quercetin remains insoluble. The mixture is then diluted 10-fold for downstream in vitro experimental testing.

For the unencapsulated control of Sample F4, 6.18 mg of quercetin (88.67% purity) is added to 20 mL of DMEM. Similar to previous observations, quercetin does not dissolve under vortex mixing or stirring, highlighting its low aqueous solubility. This formulation is also diluted 10-fold for further in vitro use.

### Solubility Comparison

The comparative water solubility between non-encapsulated phytochemicals and protein-encapsulated formulations (Samples F1-F4) is summarized in Table 2. The encapsulated prototypes exhibit a marked enhancement in aqueous solubility, with improvements ranging from 151% to as high as 382,700% for various phytochemicals including curcumin, quercetin, and ginsenoside Rg1, confirming the efficacy of the protein-zein encapsulation system in overcoming solubility limitations.

TABLE 2
The water solubility of phytochemicals with or without encapsulation at 25° C.

| | Water solubility of phytochemicals at 25° C. | | | | | | | | | | | | | |
|---|---|---|---|---|---|---|---|---|---|---|---|---|---|---|
| | | | | | F2 | | | | | | F5 | | F8 | |
| | F1 | | F2 | | (Ginsenoside | | F3 | | F4 | | (Ginsenoside | | (Tetrahydro | |
| | (Curcumin) | | (Quercetin) | | Rg1) | | (Curcumin) | | (Quercetin) | | Rg1) | | curcumin) | |
| Encapsulated | 2.32 | mg/mL | 1.07 | mg/mL | 0.77 | mg/mL | 1.74 | mg/mL | 0.66 | mg/mL | 3.16 | mg/mL | 1.04 | mg/mL |
| phytochemical | | | | | | | | | | | | | | |
| Without | 0.6 | μg/mL | 0.00215 | mg/mL | 0.304 | mg/mL | 0.6 | μg/mL | 0.00215 | mg/mL | 0.304 | mg/mL | 0.00943 | mg/mL |
| encapsulation | | | | | | | | | | | | | | |

| | | | | | | | |
|---|---|---|---|---|---|---|---|
| Solubility | 386567% | 49667% | 153% | 289900% | 30443% | 941% | 10929% |
| increase | | | | | | | |

### Example 3. In Vitro Cytotoxicity Assay

The cytotoxicity of the encapsulated phytochemical formulations is evaluated in vitro using the MTT assay with Caco-2 cells. The Caco-2 cells are cultured in high-glucose DMEM, supplemented with 10% (v/v) FBS, 1% (v/v) penicillin-streptomycin, and 1% (v/v) non-essential amino acids. Cells are seeded into 96-well plates at a density of 1×10⁴ cells per well and incubated for 24 hours at 37° C. under a 5% CO₂ atmosphere.

For samples F1 and F3, 50 mg of each sample is weighed and dissolved in 10 mL of DMEM, followed by filtration through a 0.22 μm syringe filter to obtain a working stock solution with a concentration of 5 mg/mL. Serial dilutions are subsequently performed to prepare test concentrations of 2.5, 1.25, 0.625, 0.3125, and 0.15625 mg/mL.

For samples F2 and F4, 150 mg of each sample is weighed and dissolved in 5 mL of DMEM, followed by filtration through a 0.22 μm filter to prepare a 30 mg/mL stock solution. Serial dilutions are conducted to obtain concentrations of 15, 7.5, 3.75, and 1.875 mg/mL.

Following the 24-hour incubation period, the cell culture medium is replaced with fresh medium containing the respective concentrations of each sample. Cells are treated with the samples for 2 hours at 37° C. Subsequently, the treatment media are removed, and 20 μL of MTT solution (5 mg/mL in PBS) along with 100 μL of fresh culture medium is added to each well. The plate is incubated for an additional 3 hours at 37° C. to allow for formazan crystal formation. After incubation, the medium is aspirated, and the resulting formazan crystals are solubilized using DMSO. Absorbance is measured at 595 nm using a microplate spectrophotometer.

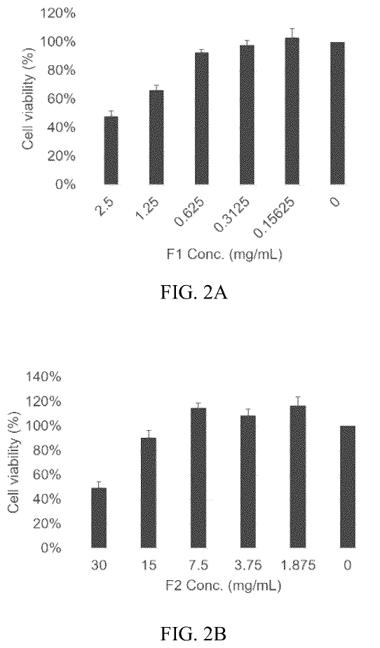

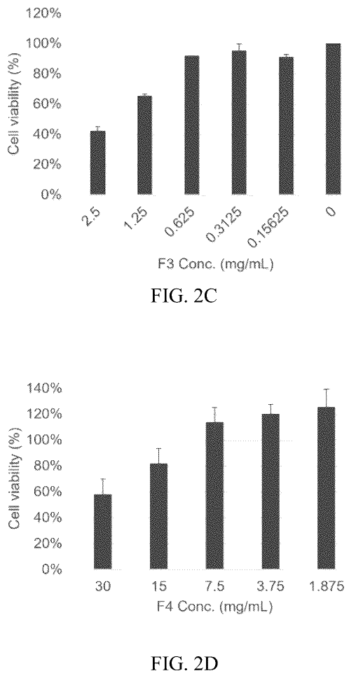

The results demonstrate that no significant cytotoxic effects are observed in Caco-2 cells treated with sample F1 or F3 at concentrations of 0.625 mg/mL or lower, or with sample F2 or F4 at concentrations of 15 mg/mL or lower (see FIGS. 2A-2D), indicating good biocompatibility of the encapsulated formulations at those tested doses.

### Example 4. In Vitro Intestinal Absorption Assay

An in vitro intestinal absorption assay is conducted using Caco-2 cell monolayers cultured on Transwell filter inserts to evaluate the transport of encapsulated phytochemicals across the intestinal epithelium. To prepare the monolayers, Caco-2 cells are seeded at a density of 6×10⁵ cells per insert and cultured for 21 days, with the medium being replaced every 2-3 days. Prior to conducting the absorption experiment, the integrity of the monolayers is assessed on day 21 by measuring TEER using a Millicell ERS-2 volt-ohm meter (Merck Millipore). Only monolayers with TEER values greater than 400 Ω·cm² are deemed suitable for use in transport studies, indicating the formation of tight junctions.

Before initiating the experiment, the apical and basolateral surfaces of the cell monolayers are washed with pre-warmed Hank's Balanced Salt Solution (HBSS) at 37° C. Then, 1 mL of fresh HBSS is added to each basolateral chamber. For the transport study, sample F1 and F3 solutions are prepared at 0.625 mg/mL, and sample F2 and F4 solutions at 15 mg/mL, by dissolving each in 200 μL of HBSS. These are added to the apical chamber of the Transwell system.

For control comparisons, unformulated solutions of raw curcumin and quercetin, respectively equivalent in concentration to the active component in F1 and F2, are also dissolved in 200 μL of HBSS and applied to separate wells. All setups are incubated at 37° C. for a period of 2 hours to allow for compound transport. Following incubation, both apical and basolateral compartment samples are collected and filtered through 0.45 μm syringe filters. The filtrates are stored at −20° C. prior to quantitative analysis via high-performance liquid chromatography coupled with mass spectrometry (HPLC-MS).

The HPLC-MS results reveal a substantial enhancement in intestinal permeability for the encapsulated phytochemicals. Specifically, curcumin transport from sample F1 shows a dramatic increase, with no detectable transport in the unformulated control—thus indicating an immeasurable fold enhancement (+∞). Similarly, quercetin absorption from sample F2 exhibits a 688.76% increase in basolateral concentration relative to the unformulated quercetin control. These findings demonstrate that the nano-encapsulation method significantly improves the intestinal absorption potential of low-solubility phytochemicals.

### Example 5. In Vitro Anti-Inflammatory Assay

To evaluate the anti-inflammatory potential of the encapsulated phytochemical formulations, RAW 264.7 murine macrophage cells are used in an in vitro lipopolysaccharide (LPS)-induced inflammation model. The cells are cultured in high-glucose DMEM supplemented with 1% (v/v) penicillin-streptomycin and 10% (v/v) FBS and maintained at 37° C. in a humidified incubator with 5% CO₂. Cells are seeded into 24-well culture plates at a density of 3×10⁵ cells per well and incubated for 24 hours.

For treatment preparation, 50 mg of each of sample F1 and F3 is dissolved in 10 mL of DMEM, filtered through a 0.22 μm membrane to ensure sterility, and diluted to a stock concentration of 5 mg/mL. Serial dilutions are then performed to generate a range of working concentrations. An unformulated curcumin control is also prepared at an equivalent curcumin concentration, following the same steps of dissolution, filtration, and dilution. After the initial incubation, the culture medium is replaced with fresh DMEM containing 0.1 mg/mL LPS to induce inflammation, with or without the respective treatment samples or controls. Cells not exposed to LPS or treatment serve as the negative control group.

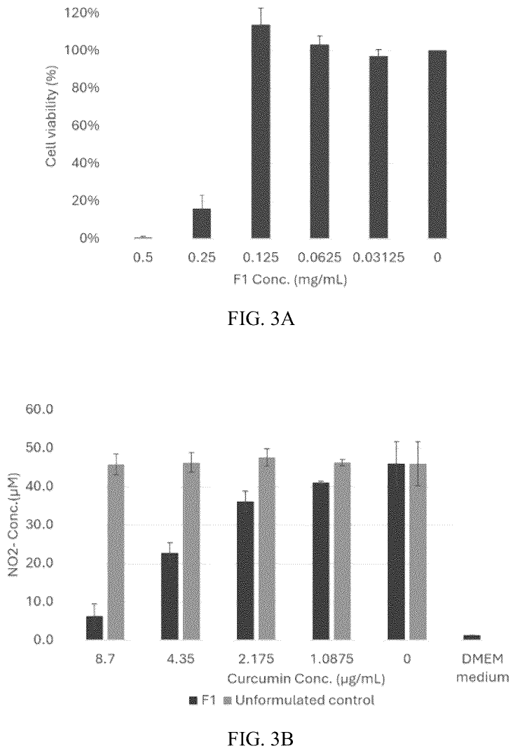

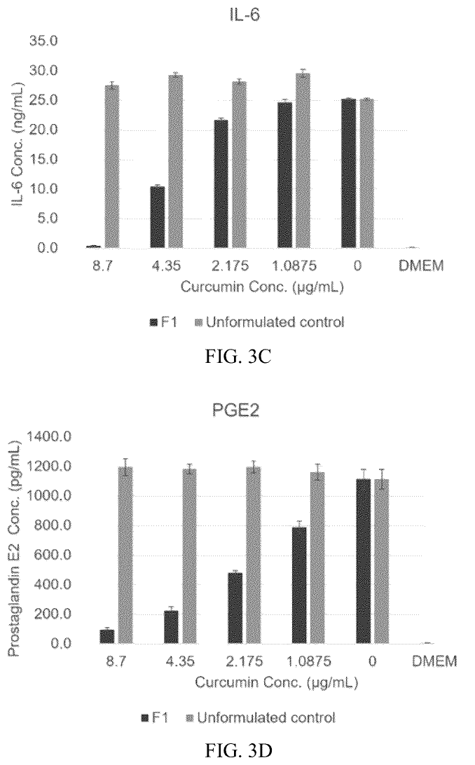

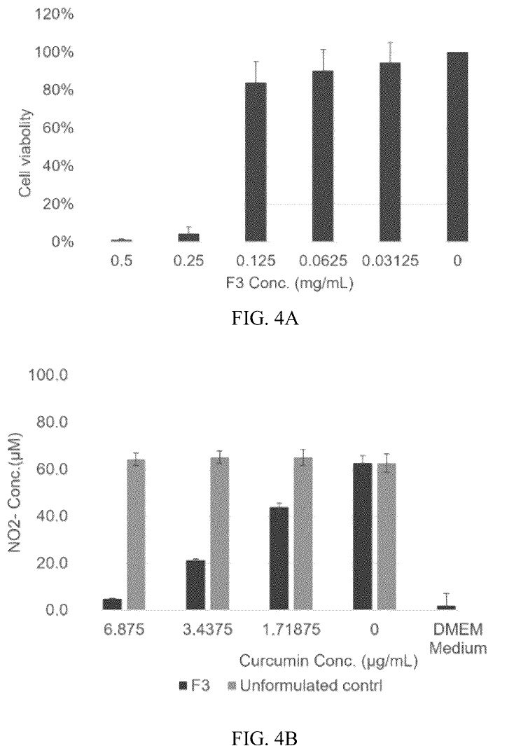

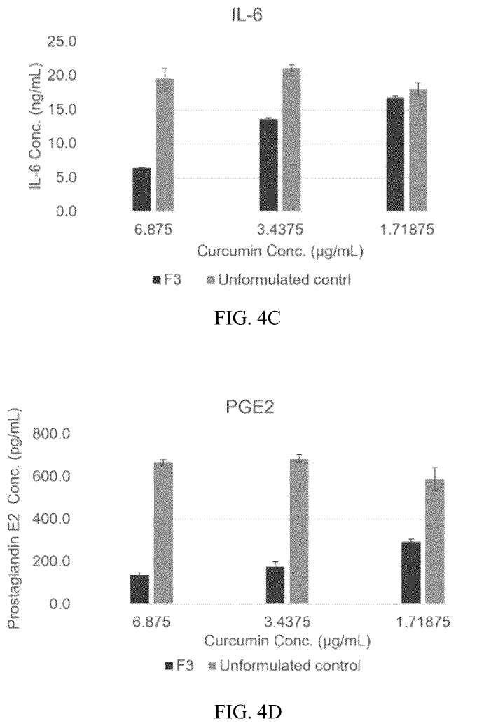

Cytotoxicity of F1 and F3 is assessed using an MTT assay, which demonstrates that concentrations up to 0.125 mg/mL show no adverse effects on cell viability (FIGS. 3A and 4A), validating the use of this concentration for anti-inflammatory evaluation.

The anti-inflammatory effects are evaluated by quantifying nitric oxide (NO) production using the Griess reagent assay, which detects nitrite (NO₂_{^{−}}), a stable metabolic end-product of NO. A total of 100 μL of cell culture supernatant from each well is transferred into a 96-well microplate, followed by the addition of 100 μL of Griess reagent. The plates are incubated in the dark for 15 minutes, and absorbance is read at 540 nm. A nitrite standard curve is used to calculate NO₂_{^{−}} concentrations.

LPS-stimulated control cells exhibit significantly elevated NO production relative to the non-LPS-treated negative control. Treatment with F1 or F3 at 0.125 mg/mL (containing 8.7 μg/mL and 6.875 μg/mL of curcumin, respectively) substantially reduces NO₂_{^{−}} levels. Specifically, NO₂_{^{−}} concentrations are reduced to 6.26±3.18 μM for F1 and 4.87±1.66 μM for F3, corresponding to anti-inflammatory inhibition rates of 86.02% and 94.9%, respectively, compared to the unformulated control (FIGS. 3B and 4B).

To further assess the anti-inflammatory response, the concentrations of interleukin-6 (IL-6) and prostaglandin E₂ (PGE₂) in the culture supernatants are measured using commercial ELISA kits in accordance with the manufacturers' protocols.

The levels of IL-6 and PGE₂ in the LPS-only and unformulated control groups are markedly elevated, indicating successful induction of inflammation (FIGS. 3C-3D and 4C-4D). Treatment with F1 and F3 at 0.125 mg/mL results in a significant reduction in pro-inflammatory cytokine production. Specifically, IL-6 levels are reduced by 107.74% and 67.28%, and PGE₂ levels are decreased by 99% and 79.69%, respectively, when compared to the unformulated control groups (Table 3 and Table 4). These findings confirm the superior anti-inflammatory effects of the protein-encapsulated curcumin formulations relative to free curcumin.

TABLE 3
Concentration of curcumin in F1 formulation

| F1 Formulation | Amount of Curcumin |
|---|---|
| (mg/mL) | in F1 (μg/mL) |
| 0.125 | 8.7 |
| 0.0625 | 4.35 |
| 0.03125 | 2.175 |
| 0.015625 | 1.0875 |

TABLE 4
Concentration of curcumin in F3 formulation

| F3 Formulation | Amount of Curcumin |
|---|---|
| (mg/mL) | in F3 (μg/mL) |
| 0.125 | 6.875 |
| 0.0625 | 3.4375 |
| 0.03125 | 1.71875 |

### Example 6. In Vitro Anti-Adipogenic Assay

To evaluate the anti-adipogenic properties of the encapsulated phytochemical prototypes, 3T3-L1 preadipocyte cells are cultured in 12-well plates at a density of 4×10⁴ cells per well. The cells are maintained in high-glucose DMEM supplemented with 1% (v/v) penicillin-streptomycin and 10% (v/v) FBS, under standard culture conditions at 37° C. in a humidified 5% CO₂ atmosphere.

For treatment preparation, 40 mg each of samples F2 and F4 are weighed and dissolved separately in 10 mL of DMEM. The resulting solutions are filtered through a 0.22 μm membrane to ensure sterility, yielding stock concentrations of 4 mg/mL. Serial dilutions are subsequently prepared to generate a range of working concentrations for experimental use. For comparison, an unformulated control containing equivalent amounts of quercetin and ginsenoside Rg1 is prepared using the same procedure of dissolution, filtration, and dilution.

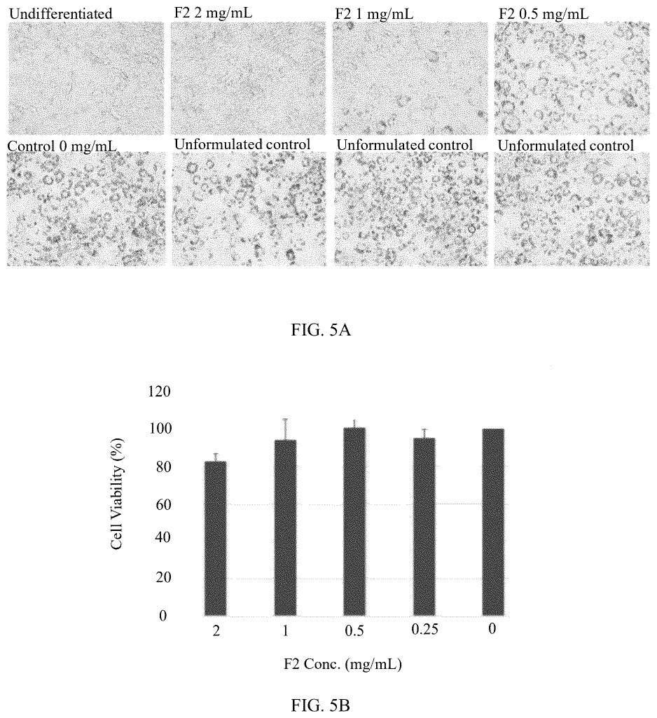

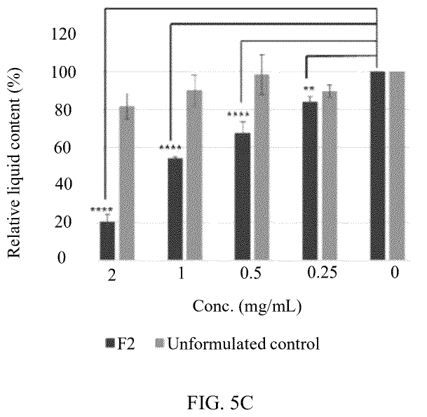

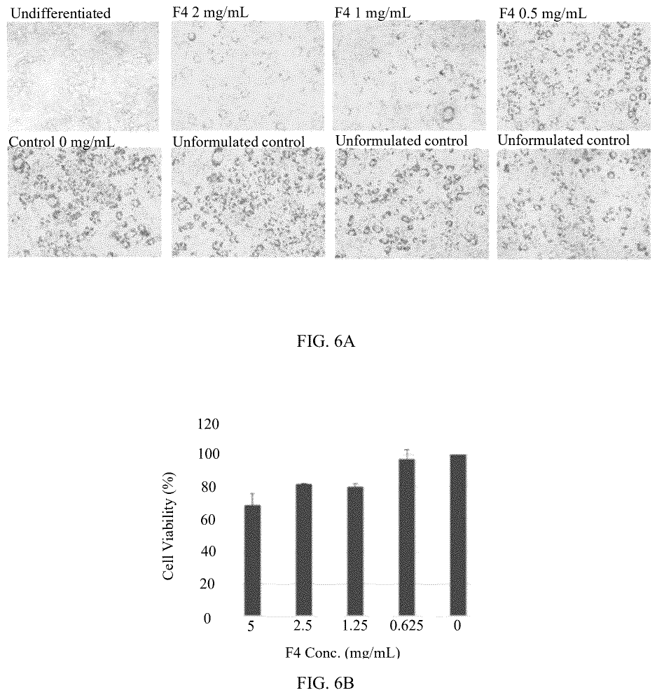

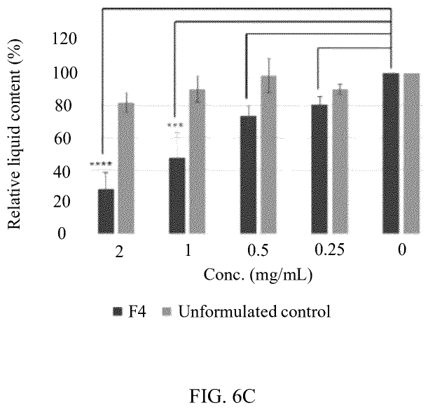

Once the 3T3-L1 cells reach 80% to 100% confluency, the medium is replaced with an adipogenic induction medium containing 0.5 mM isobutylmethylxanthine (IBMX), 1 μM dexamethasone, and 10 μg/mL insulin (referred to as MDI medium). The cells are incubated in this medium for 2 days at 37° C. in a 5% CO₂ environment in the presence of either sample F2 or the unformulated control. Subsequently, the induction medium is replaced with maintenance medium consisting of DMEM supplemented with 1% (v/v) penicillin-streptomycin, 10% (v/v) FBS, and 10 μg/mL insulin, and cells are incubated for an additional 3 days. On the 7th day, the medium is again changed to fresh DMEM containing 1% (v/v) penicillin-streptomycin and 10% (v/v) FBS. Adipocyte-like differentiation us observed on day 8 (see FIGS. 5A and 6A).

To assess cytotoxicity prior to anti-adipogenic analysis, an MTT assay is performed. No cytotoxic effects are observed for F2 at concentrations up to 2 mg/mL or for F4 at concentrations up to 2.5 mg/mL, confirming these doses are suitable for downstream analysis of anti-adipogenic activity (FIGS. 5B and 6B).

Intracellular lipid accumulation in differentiated 3T3-L1 cells is assessed on day 8 using Oil Red O staining. After aspirating the culture medium, the cells were rinsed twice with PBS and fixed in 4% paraformaldehyde (PFA). Following fixation and additional PBS washes, the cells are stained with Oil Red O for 10 minutes, then washed three times with distilled water. Microscopic evaluation is performed at 200× magnification. Untreated cells serve as the negative control. For quantitative analysis, the retained stain is extracted using isopropanol, and absorbance is measured at 495 nm using a microplate reader. The percentage of lipid accumulation is calculated as the ratio of absorbance in the sample-treated or control groups to that of the untreated control group.

Microscopic observation reveals that cells in the control group develop large intracellular lipid droplets, indicative of adipogenic differentiation, while untreated cells maintain a fibroblast-like morphology with minimal lipid content. Treatment with F2 and F4 notably suppresses lipid accumulation in a dose-dependent manner. At a concentration of 2 mg/mL, sample F2 reduces intracellular lipid droplet content to 20.20±4.23%, while sample F4 reduces it to 27.87±10.27%. These reductions correspond to improvements in anti-adipogenic activity of 75.24% and 65.84%, respectively, relative to the unformulated control (see FIGS. 5C, FIG. 6C, Table 5 and Table 6).

TABLE 5
Concentration of ginsenoside Rg1 in F2

| F2 Formulation | Amount of Ginsenoside | Amount of Quercetin |
|---|---|---|
| (mg/mL) | Rg1 in F2 (μg/mL) | in F2 (μg/mL) |
| 2 | 46.2 | 64.2 |
| 1 | 23.1 | 32.1 |
| 0.5 | 11.55 | 16.05 |
| 0.25 | 5.775 | 8.025 |

TABLE 6
Concentration of quercetin in F4

| F4 Formulation | Amount of Quercetin |
|---|---|
| (mg/mL) | in F4 (μg/mL) |
| 2 | 27.4 |
| 1 | 13.7 |
| 0.5 | 6.85 |
| 0.25 | 3.425 |

### Example 7. In Vivo Bioavailability Evaluation

Sample F1 is evaluated for in vivo bioavailability in a rat model. The study includes a total of ten rats, comprising five males and five females. Each rat is administered Sample F1 orally at a dosage of 20 mL/kg, with the formulation containing 143.5 mg/mL of curcumin, resulting in a total administered dose of 2.87 g/kg. Blood samples are collected at eight predefined time points: 15 minutes, 30 minutes, 1 hour, 2 hours, 3 hours, 4 hours, 8 hours, and 24 hours post-administration. Plasma curcumin concentrations are then analyzed using liquid chromatography-tandem mass spectrometry (LC-MS/MS), and the results are summarized in Table 7. A control group, treated in the same manner with unformulated curcumin (UC), serves as the reference. Notably, curcumin is undetectable in the plasma of all rats in the control group at every time point.

TABLE 7
The curcumin concentration in plasma

| | Male | | | Female | | |
|---|---|---|---|---|---|---|
| | Mean | | | Mean | | |
| | concentration | | | concentration | | |
| | of curcumin | | | of curcumin | | |
| Time | in rat plasma | SD | RSD | in rat plasma | SD | RSD |
| (h) | (ng/mL) | (ng/mL) | % | (ng/mL) | (ng/mL) | % |
| 0.25 | 100 | 109 | 108.4 | 49.6 | 15.8 | 31.9 |
| 0.5 | 63.1 | 46.8 | 74.3 | 21.5 | 11.3 | 52.7 |
| 1 | 19.0 | 9.39 | 49.4 | 29.0 | 22.2 | 76.5 |
| 2 | 20.4 | 16.6 | 81.6 | 21.8 | 14.9 | 68.1 |
| 3 | 17.9 | 12.7 | 70.9 | 22.9 | 11.7 | 51.2 |
| 4 | 20.0 | 11.6 | 57.9 | 18.5 | 15.5 | 83.8 |
| 8 | 5.66 | 6.07 | 107.3 | 5.28 | 3.8 | 71.5 |
| 24 | NA | NA | NA | NA | NA | NA |

The average pharmacokinetic parameters of curcumin after a single oral administration of F1 and UC in rats are presented in Table 8. In the F1 group, the AUC₀₋ₜ of curcumin is 179±35.4 hng/mL for male rats and 159±51.5 hng/mL for female rats. However, in the UC group, the AUC 0-t of curcumin is not detected in either male or female rats.

TABLE 8
Average pharmacokinetic parameters of curcumin after single
oral administration of F1 and unformulated curcumin in rats

| | | Male | | Female | |
|---|---|---|---|---|---|
| Pharmacokinetic | | | Unformulated | | Unformulated |
| parameters | | F1 | curcumin | F1 | curcumin |
| t½ | (h) | 1.9 ± 0.356 | N.D. | 1.9 ± 0.378 | N.D. |
| Tmax | (h) | 0.3 ± 0.112 | N.D. | 1.15 ± 1.63 | N.D. |
| Cmax | (ng/mL) | 106 ± 108 | N.D. | 54 ± 15.6 | N.D. |
| AUC 0-t | (h*ng/mL) | 152 ± 46.2 | N.D. | 144 ± 44.1 | N.D. |
| AUC 0-∞ | (h*ng/mL) | 179 ± 35.4 | N.D. | 159 ± 51.5 | N.D. |

*N.D. = Not Detected

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or 0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within ±10%, 5%, ±1%, or ±0.5% of the average of the values.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A method for encapsulating a low-solubility phytochemical to form a water-soluble nano-complex, comprising:
creating a mixture by combining one or more proteins and a solvent;
adjusting a pH of the mixture to an alkaline condition by adding an alkaline compound;
sequentially adding zein protein and a low-solubility phytochemical into the mixture with mixing, wherein the zein protein unfolds under the alkaline condition to capture the low-solubility phytochemical through hydrophobic interactions;
adjusting the pH of the mixture to an approximately neutral pH value by adding an acidic compound to induce refolding of the zein protein to fix the low-solubility phytochemical within a nano-complex, forming a nano-complex solution; and
drying the nano-complex solution to produce a dry powder form of the nano-complex;
wherein the nano-complex comprises the one or more proteins encapsulating the low-solubility phytochemical.

2. The method of claim 1, wherein the nano-complex has a particle size less than 1 μm.

3. The method of claim 1, further comprising heating the mixture to a temperature between 80° C. and 99° C. before the addition of the low-solubility phytochemical and zein protein;
wherein the heating is applied for a duration of 5 to 30 minutes.

4. The method of claim 1, wherein the low-solubility phytochemical comprises one or more of curcumin, quercetin, ginsenosides, tea polyphenols, and tetrahydrocurcumin.

5. The method of claim 1, wherein the one or more proteins are water-soluble and selected from the group consisting of animal proteins, plant-based proteins, and combinations thereof.

6. The method of claim 1, wherein the alkaline compound is sodium hydroxide, potassium hydroxide or a combination thereof.

7. The method of claim 1, wherein the alkaline condition is a condition with a pH value greater than 11.

8. The method of claim 1, wherein the approximately neutral pH value is a pH value ranges from 5 to 8.

9. The method of claim 1, wherein the acidic compound is hydrochloric acid, citric acid, ascorbic acid, lactic acid, malic acid, acetic acid or a combination thereof.

10. The method of claim 1, wherein the drying of the nano-complex solution is performed using freeze drying, convection drying, or spray drying.

11. The method of claim 1, wherein the protein encapsulation increases the solubility of the low-solubility phytochemical by at least 150%.

12. The method of claim 1, wherein the nano-complex has a particle size ranging from 40 to 200 nanometers.

13. The method of claim 5, wherein the one or more proteins are selected from the group consisting of casein, whey protein, soy protein, and pea protein.

14. A water-soluble encapsulated phytochemical complex powder fabricated by the method of claim 1, comprising:
a water-soluble nano-complex, comprising:
an encapsulation layer, comprising the one or more proteins; and
a core, comprising the zein protein and the low-solubility phytochemical, wherein the zein protein and the low-solubility phytochemical are bound together through hydrophobic interactions.

15. The complex powder of claim 14, wherein the water-soluble nano-complex has a particle size less than 1 μm.

16. The complex powder of claim 14, wherein the particle size of the water-soluble nano-complex ranges from 40 to 200 nanometers.

17. The complex powder of claim 14, wherein the encapsulated phytochemical exhibits increased water solubility and bioavailability compared to its unencapsulated form.

18. The complex powder of claim 14, wherein the low-solubility phytochemical comprises one or more of curcumin, quercetin, ginsenosides, tea polyphenols, and tetrahydrocurcumin.

19. The complex powder of claim 14, wherein the encapsulation layer is formed from a combination of plant-based proteins and animal-based proteins; and
wherein the encapsulation layer provides physical protection and oxidative stability to the low-solubility phytochemical.

20. The complex powder of claim 14, wherein the encapsulated phytochemical is used in pharmaceutical, nutraceutical, cosmetic or food supplement applications.
